(19) United States
(12) Patent Application Publication
(10) Pub. No.: US 2017/0340691 A1
(43) Pub. Date: Nov. 30, 2017

(54) An Antimicrobial Composition

(71) Applicant: Neem Biotech Limited, Abertillery (GB)

(72) Inventors: Carl Jorg Michael GRAZ, Abertillery (GB); Gareth James Street EVANS, Abertillery (GB); Robert Alun SAUNDERS, Abertillery (GB); Liam James JONES, Abertillery (GB)

(73) Assignee: Neem Biotech Limited, Abertillery (GB)

(21) Appl. No.: 15/533,628

(22) PCT Filed: Dec. 15, 2015
(86) PCT No.: PCT/GB15/54018

§ 371 Date: Jun. 6, 2017

(30) Foreign Application Priority Data
Dec. 23, 2014 (GB) 1423115.3

(51) Int. Cl. A61K 36/88; A61K 31/352; A61K 31/194; A61K 31/7048; A61K 31/10

(52) U.S. Cl. CPC A61K 36/88; A61K 31/7048; A61K 31/10; A61K 31/194; A61K 31/352

(57) ABSTRACT

The present invention provides an antimicrobial composition comprising at least one garlic extract and one or more of a bioflavonoid and/or an organic acid as well as the use of the same for treating or preventing bacterial, fungal or parasitic infections.

### FIELD OF INVENTION

The present invention relates to antimicrobial compositions, as well as to uses of the same and methods for treating or preventing bacterial, fungal or parasitic infections.

### BACKGROUND TO THE INVENTION

The worldwide problem of bacterial resistance has grown to such an extent that several organisms (for example, Klebsiella pneumoniae, Escherichia coli and Staphylococcus aureus) have developed resistance to multiple different antibiotics. According to the Centres for Disease Control and Prevention (CDC), in 2013 there were over 2 million infections with antibiotic resistant microorganisms and over 23 thousand subsequent deaths from those infections. A little under half of those deaths (11,000) occurred as a result of methicillin-resistant S. aureus (MRSA). Another organism that was responsible for a large number of deaths, (14,000, even higher than MRSA) in 2013, was Clostridum difficile. This bacterium has proven to be very resistant to antibiotics. However, the resistance of C. difficile is due to its ability to produce spores, rather than specific strain resistance (hence not being included in the year total for antibiotic resistance infection deaths).

Combinations of antimicrobials with improved efficacy are much sought after in the present clinical industry. The combination antibiotic therapy is used in an attempt to broaden the bacterial spectral range, and thus to avoid the emergence of resistance or multi-resistance and lead to a better clinical outcome. Understanding of the mechanisms of antimicrobial actions and researches on potential antimicrobial agents are vital in the development of an effective combination of antimicrobials.

In view of the growing problem of antibiotic resistance, there is a need for novel antibiotic compositions that are effective against a wide variety of microorganisms, especially antibiotic resistant microorganisms.

### SUMMARY OF INVENTION

The present invention provides a novel combination of garlic compounds with bioflavonoids and/or organic acids, which can provide an antimicrobial effect, preferably a synergistic antimicrobial effect, against broad-spectrum of microorganisms, including bacteria, fungi and parasites.

The present invention provides an antimicrobial composition comprising at least one garlic extract and one or more of a bioflavonoid and/or an organic acid. The composition can comprise at least one garlic extract and at least one bioflavonoid, or at least one garlic extract and at least one organic acid. The composition may also include at least one garlic extract, at least one bioflavonoid and at least one organic acid.

The concentration ratio of the garlic extract to the bioflavonoid can be about 1-16:16-1. For example, the concentration ratio of the garlic extract to the bioflavonoid is about 1-4:4-1. Similarly, the concentration ratio of the garlic extract to the organic acid can be about 1-16:16-1, for example, the concentration ratio of the garlic extract to the organic acid is about 1-4:4-1. For a composition comprises at least one garlic extract, at least one bioflavonoid and at least one organic acid, the concentration ratio of the garlic extract to the bioflavonoid and to the organic acid can be about 1-16:1-16:1-16. Alternatively, the concentration ratio of the garlic extract to the bioflavonoid and to the organic acid can be about 1-4:1-4:1-4.

The garlic extract can be selected from one or more of crude garlic, allicin and ajoene; whilst, the bioflavonoid can be selected from one or more of the group consisting of flavones, flavonols, flavanones, flavanone glycosides, flavanonols, flavans and anthocyanidins. These bioflavonoids can be one or more of acacetin, rhoifolin luteolin, apigenin, tangeritin, quercetin, kaempferol, myricetin, fisetin, galangin, isohamnetin, pachypodol, rhamnazin, hesperetin, naringenin, eriodictyol, homoeriodictyol, naringin, hesperidin, neohesperidin, catechin, cardamonin, procyanidin, sillibinin, genistein or any combination thereof. The organic acid used in the present invention can be selected from one or more of carboxylic acids and sulfonic acids, such as lactic acid, butyric acid, propionic acid, valeric acid, caproic acid, acetic acid, formic acid, citric acid, oxalic acid, sorbic acid, benzoic acid, caprylic acid and malic acid, or any combination thereof.

The present invention provides an antimicrobial composition which may further comprise a pharmaceutically acceptable carrier and/or excipient. The composition can be formulated for oral, topical, intravenous, intramuscular, intrarectal (suppository), inhalant, infusion, transdermal, sublingual, subcutaneous or intranasal administration.

The antimicrobial composition may be an antibacterial composition, an antifungal composition or an antiparasitic composition. The composition may be used to treat or prevent bacterial infections such as Gram-negative or Gram-positive bacterial infections, fungal infections, or parasitic infections, as appropriate.

The present invention provides a method of treating or preventing a bacterial infection, wherein the method comprises administering an antimicrobial composition comprising at least one garlic extract and one or more of a bioflavonoid and/or an organic acid. The present invention also provides a method of treating or preventing a fungal infection comprising administering an antimicrobial composition comprising at least one garlic extract and one or more of a bioflavonoid and/or an organic acid. The present invention also provides a method of treating or preventing a parasitic infection comprising administering an antimicrobial composition comprising at least one garlic extract and one or more of a bioflavonoid and/or an organic acid.

The present invention also discloses the use of an antimicrobial composition comprising at least one garlic extract and one or more of a bioflavonoid and/or an organic acid in the manufacture of a medicament for treating or preventing a bacterial infection in a subject, or in the manufacture of a medicament for treating or preventing a fungal infection in a subject, or in the manufacture of a medicament for treating or preventing a parasitic infection in a subject.

### DETAILED DESCRIPTION

The combination of garlic extract with a bioflavonoid and/or an organic acid as provided in compositions of the present invention has been found to produce a surprising and unexpected synergistic action against pathogenic microorganisms. This synergistic effect of compositions of the present invention has been determined based on the antimicrobial activity of the compositions. In particular, synergy has been determined where the antimicrobial activity of a composition containing a mixture of two or more components is higher than the combined antimicrobial activity of each component alone.

Without being bound by theory, it is believed that the modes of antimicrobial action of each component underlie the antimicrobial synergy observed when the components are combined. For example, garlic compounds such as allicin have an oxidising effect on the surface of microbes. Ajoene, however, acts intracellularly by affecting enzyme function. Bioflavonoids are taken up across cell membranes and act intracellularly. Bioflavonoids are known to have enzyme-inhibiting activity, they inhibit energy metabolism and it is further postulated that they cause damage to cell membranes, leading to the inhibition of macromolecules.

Organic acids, on the other hand, are able to cross cell membranes, where they alter intracellular pH. For instance, formic acid inhibits enzymatic activity, especially that of decarboxylase; acetic acid inhibits enzymatic activity and increases heat sensitivity; propionic acid influences membrane transport inhibition on synthesis of some amino acids; lactic acid inhibits enzymatic activity; sorbic acid and benzoic acid also inhibit enzymatic activity, amino acid uptake, (inducing cell membrane-damage) and synthesis of RNA and/or DNA; caprylic acid integrates with the cell wall due to its lipophilic character and leads to subsequent cell leaking.

The synergistic antimicrobial activity of compositions of the present invention may provide an improved treatment of pathogenic microorganisms and may play an important role in fighting the growing problem of antibiotic resistance.

Antimicrobial compositions of the present invention may kill and/or inhibit the growth of microorganisms including bacteria, fungi, algae, protozoa, viruses and sub-viral agents. The compositions may be microbicidal or microbiostatic and can be disinfectants, antiseptics or antibiotics. Antimicrobial compositions of the invention may be antibacterial, antifungal or antiparasitic.

Garlic, also known as Allium sativum, is a species in the onion genus. Garlic extracts used in the present invention can include one or more of crude garlic, allicin, ajoene or a combination thereof. The garlic extracts can also be diallyl disulphide (DADS) or S-allyl cysteine (SAC). Crude garlic may be, for example, in a fresh or crushed form and encompasses garlic juice, pulp, infusion, cutting, distillate, residue, pressing or pomace.

Allicin (2-Propene-1-sulfinothioic acid S-2-propenyl) is a highly chemically reactive sulphur-containing compound derived from garlic and other Allium species. Allicin is not found natively within garlic but it is produced when the cells are damaged and the enzyme alliinase (alliin lyase) is released from the vacuoles, coming into contact with the compound alliin ((2R)-2-amino-3-[(S)-prop-2-enylsulfinyl]propanoic acid) and converting it in to allicin. Suitable allicin for use in compositions of the present invention may be obtained from a natural, synthetic or semi-synthetic source.

Ajoene ((E,Z)-4,5,9-trithiadodeca-1,6,11-triene 9-oxide), is another sulphur based compound, which can form two different isomers (E & Z). It may be used in the present invention as a mixture of both isomers or may be pure E- or Z-ajoene. Ajoene is derived from the thermal degradation of allicin under specific conditions.

Compositions of the invention preferably comprise at least one garlic extract and least one bioflavonoid, or at least one garlic extract and at least one organic acid, or at least one garlic extract, at least one bioflavonoid and at least one organic acid.

Compositions of the invention may comprise particular ratios of components. For example, the ratio of garlic extract to bioflavonoid may be about 1-16:1-16, preferably 1-4:1-4. Similarly, the ratio of garlic extract to organic acid may be about 1-16:1-16, preferably 1-4:1-4. In an embodiment of the invention, the ratio of the garlic extract to the bioflavonoid and to the organic acid may be about 1-16:1-16:1-16, preferably, about 1-4:1-4:1-4. One example of composition among garlic extract, bioflavonoid and organic acid has a concentration ratio of 1:4:2.

Bioflavonoids (also known as flavonoids) are naturally occurring polyphenol compounds produced by plants. Chemically, bioflavonoids have the structure of a 15-carbon skeleton, consisting of two phenyl rings and a heterocyclic ring and are subdivided into categories based on their basic structure. Suitable bioflavonoids for use in the present invention include anthoxanthins, such as flavones, flavonols, flavanones (including flavanone-glycosides), flavanonols, flavan, anthocyanidins or any combination thereof. Alternatively, the bioflavonoids can also be isoflavonoids or neoflavonoids.

Suitable flavones for use in the present invention include acacetin, rhoifolin (apigenin 7-O-neohesperidoside), luteolin, apigenin and tangeritin. Flavonols include quercetin, kaempferol, myricetin, fisetin, galangin, isohamnetin, pachypodol, rhamnazin, pyranoflavonols and furanoflavonols. Flavanones include hesperetin, naringenin, eriodictyol and homoeriodictyol, as well as the flavanone-glycosides such as naringin, hesperedin and neohesperidin.

Preferably, compositions of the present invention comprise one or more of acacetin, rhoifolin luteolin, apigenin, tangeritin, quercetin, kaempferol, myricetin, fisetin, galangin, isohamnetin, pachypodol, rhamnazin, hesperetin, naringenin, eriodictyol, homoeriodictyol, naringin, hesperidin, neohesperidin, catechin, cardamonin, procyanidin, sillibinin, genistein or a combination thereof.

Compositions of the invention may comprise allicin with one or more of a flavone, a flavonol, a flavanone, a flavanonol, a flavan and an anthocyanidin, or a combination thereof. Alternatively, compositions of the invention may comprise ajoene with one or more of a flavone, a flavonol and a flavanone, a flavanonol, a flavan and an anthocyanidin, or a combination thereof. In embodiments of the invention the compositions may additionally include one or more organic acids.

Organic acids are organic compounds having acidic properties and include carboxylic acids and sulfonic acids. Suitable organic acids for use in compositions of the present invention include, but are not limited to, lactic acid, butyric acid, propionic acid, valeric acid, caproic acid, acetic acid, formic acid, citric acid, oxalic acid, sorbic acid, benzoic acid, caprylic acid and malic acid, or any combination thereof.

Compositions of the present invention may additionally include one or more pharmaceutically acceptable carriers and/or excipients, such as diluents, adjuvants, excipients, vehicles, fillers, binders, disintegrating agents, wetting agents, emulsifying agents, suspending agents, perfuming agents, buffers, dispersants, thickeners, solubilising agents, lubricating agents and dispersing agents, depending on the nature of the mode of administration and dosage forms.

The compositions may take the form, for example, of solid preparations including tablets, capsules, drageés, lozenges, granules, powders, pellets and cachets; and liquid preparations including gels, lotions, suspensions, elixirs, syrups, suspensions, sprays, emulsions and solutions.

The composition of the present invention may be administered in the form of a composition comprising any suitable additional component, such as an additional antimicrobial agent, a nutraceutical, or a dietary supplement.

The composition of the present invention can also contain an additive, such as flavouring agents, colourants, stabilizers, preservatives, artificial and natural sweeteners and the like.

Compositions of the present invention may be for use in treating or preventing a bacterial infection. The bacterial infection may be a Gram-positive bacterial infection or a Gram-negative bacterial infection, or a combination thereof. Gram-positive bacteria include, for example, Streptococci, such as S. viridans, Staphylococci, such as S. aureus, and Bacillus, such as B. subtilis, B. anthracis and B. cereus.

Gram-negative bacteria include, for example, E. coli, Pseudomonas, such as P. aeruginosa, and Klebsiella, such as K. pneumonia, K. aerogenes and K. oxytoca. Preferably, the infection is a Gram-negative bacterial infection. The composition can also be for use in treating or preventing Gram-positive bacterial infection.

Compositions of the present invention may be for use in treating or preventing a fungal infection. The fungal infection may be a Candida infection, for example, an infection with C. albicans, C. parapsilosis or C. tropicalis, or a combination thereof.

The present invention also provides methods of treating or preventing bacterial, fungal or parasitic infections, the methods comprising administering an antimicrobial composition of the invention to a patient in need thereof.

The composition may be formulated for oral, topical, intravenous, intramuscular, intrarectal (suppository), transdermal, sublingual, subcutaneous or intranasal administration. Preferably the composition is formulated for oral or topical administration.

In one embodiment of the present invention, the composition can be combined with a food or foodstuff before oral consumption. The solid or liquid form preparations may be mixed into the food or foodstuff or applied to the food, foodstuff or feed of a subject. Such solid forms include powders, granules, pellets and the like.

In certain embodiments, the subject is a human, a primate, bovine, ovine, equine, porcine, avian, rodent (such as mouse or rat), feline, or canine. Preferably, the subject is a human. The subject can also be production animals such as cattle, oxen, deer, goats, sheep and pigs, working and sporting animals such as dogs, horses and ponies, companion animals such as dogs and cats, and laboratory animals such as rabbits, rats, mice, hamsters, gerbils or guinea pigs.

The present invention also discloses use of an antimicrobial composition comprising at least one garlic extract and one or more of a bioflavonoid and/or an organic acid in the manufacture of a medicament for treating or preventing a bacterial infection in a subject, or in the manufacture of a medicament for treating or preventing a fungal infection in a subject, or in the manufacture of a medicament for treating or preventing a parasitic infection in a subject.

The antimicrobial composition as embodied in the present invention can be useful as a treatment or prophylaxis against common pathological microorganisms, especially those widely distributed and can be commensals of the body of human or mammals. These microorganisms can include the pathogens present in the human or mammals' upper respiratory tracts, gastrointestinal tracts, oral cavities, skin, urinary tracts, or female genital tracts.

The present disclosure includes features as contained in the appended claims, as well as that of the foregoing description. Although this invention has been described in its preferred form with a degree of particularity, it is understood that the present disclosure of the preferred form has been made only by way of example and that numerous changes in the details of construction and the combination and arrangements of parts may be resorted to, without departing from the scope of the invention.

### BRIEF DESCRIPTION OF THE DRAWINGS

The invention is now described in specific embodiments with reference to accompanying drawings in which:

FIG. 1 shows the response of Gram-positive bacteria (B. subtilis, S. aureus and S. viridans) (1A), Gram-negative bacteria (E. coli, P. aeruginosa and K. pneumonia) (1B) and yeast (C. tropicalis) (1C) to allicin, as well as the response of bacteria (B. subtilis, S. aureus, S. viridans, E. coli, P. aeruginosa and K. pneumonia) (1D) and yeast (C. tropicalis) (1E) to ajoene.

FIG. 2 shows the structures of rhoifolin (A), acacetin (B), naringin (C), narangenin (D), quercetin (E), allicin (F) and ajoene (G).

### EXAMPLES

Examples are provided below to illustrate different aspects and embodiments of the present invention. These examples are not intended in any way to limit the disclosed invention, which is limited only by the claims.

### Example 1

### Synergistic Test between Garlic Extracts and Bioflavonoids

A synergistic test was conducted to test whether the antimicrobial efficacy of allicin and ajoene can be enhanced synergistically by polyphenols in the flavone (such as rhoifolin), flavonol (such as quercetin), flavanone (such as naringenin) and flavanone glycoside (such as naringin) groups of bioflavonoids.

The presence of synergy was determined by subtracting individual inhibition results for each compound from a mixed inhibition result for the corresponding compounds and concentrations. Any result greater than 0 was considered to be synergistic because the combination has a greater effect than the sum of its individual parts.

In order to determine the minimal inhibitory concentration (MIC) for the bioflavonoids (BC), allicin (AL) and ajoene (AJ) on selected Gram-positive bacteria, Gram-negative bacteria and yeast, a modified broth dilution assay using 96-well plates, according to Wiegand, Hilpert and Hancock (2008), was performed.

### Example 2

### Preparation of Microbial Cultures

For testing purposes, a Gram-positive species (S. aureus), three Gram-negative species (E. coli, P. aeruginosa and K. pneumonia) and a yeast (C. tropicalis) were used.

The microorganisms for the study were obtained from Cardiff University. The cultures used in the study were prepared by aseptic inoculation of microorganisms from pure plate cultures into 50 ml of sterile nutrient broth. The cultures were incubated overnight at 37.0° C. without agitation. Prior to use the cultures were washed by centrifugation in a desk top centrifuge at 3000 RPM for 15 minutes and the pellet resuspended in sterile saline (0.85% NaCl).

### Example 3

### Preparation of Compounds

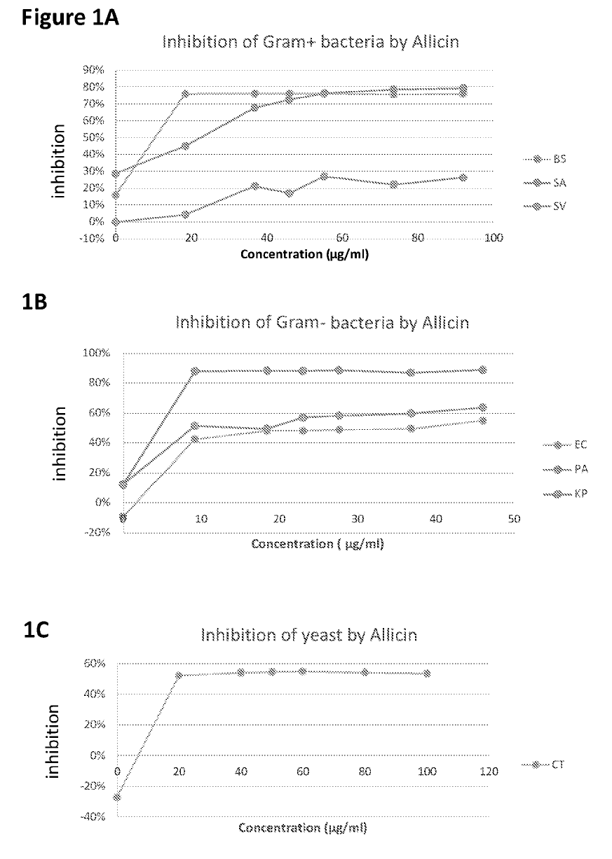

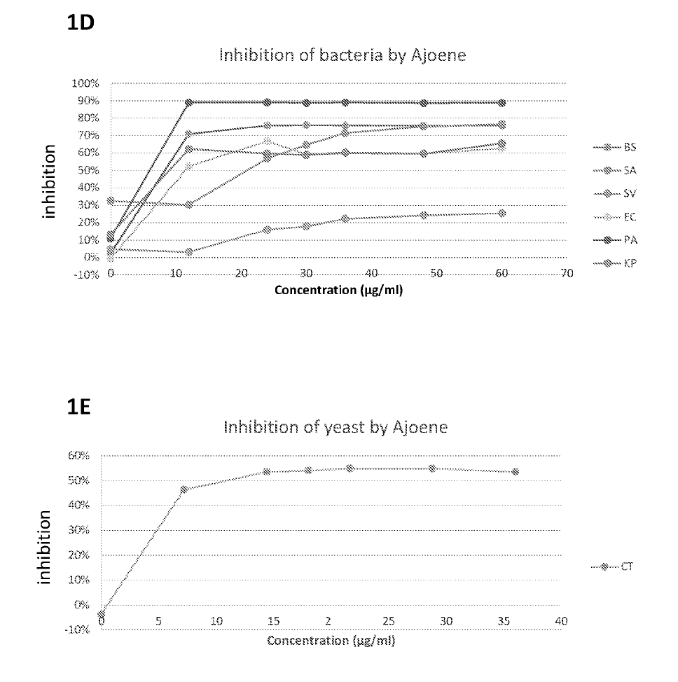

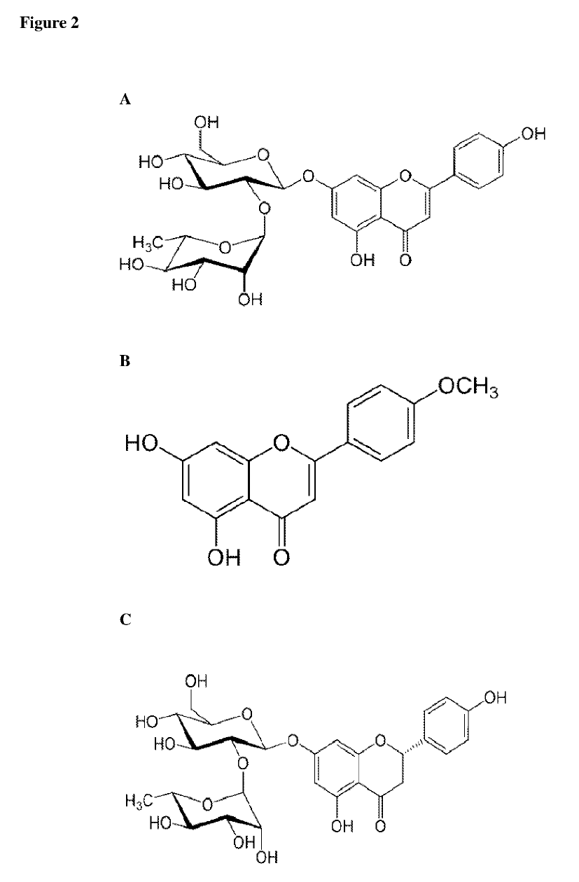

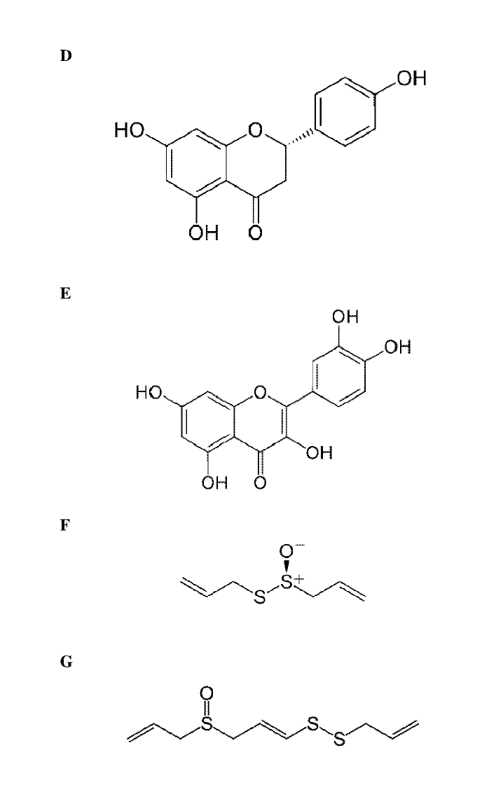

In order to determine the activity of the compounds both individually as well as in combination, a range of concentrations of each compound was used in a doubling dilution. The highest concentration used was dependant on the organism in question (whether Gram-positive bacteria, Gram-negative bacteria or yeast). From the highest concentration used in the test the samples were diluted five times in order to give six different concentrations per compound. The compounds were all tested individually as well as in a matrix of concentrations including mixtures of AL:BC, AJ:BC and AL:AJ:BC. The dilution gradients of AL and AJ were run inversely to BC (as shown in FIG. 1A-H).

The identities of the garlic compounds were confirmed by HPLC analysis using a calibrated method. The Bioflavonoids were purchased from Sigma-Aldrich and were supplied with Certificates of Analysis to confirm their identity.

The allicin samples were aqueous solutions and were therefore diluted directly using H₂O. However, the ajoene was in oil form and the bioflavonoids were in powder form, neither of which were water soluble. Before dilution the ajoene and bioflavonoids were solubilised by creation of a stock solution using 80% DMSO.

### Example 4

### Preparation of Plates

The wells on the 96-well plates were prepared in triplicate as follows: the AL, AT and BC were pre-diluted in Eppendorf tubes at 10× the required final concentration. 20 μl of each dilution was added per well to give a 1:10 dilution in the final 200 μl per well. A matrix of concentrations of mixtures of allicin:bioflavonoid, ajoene:bioflavonoid and allicin:ajoene:bioflavonoid was used, with the dilution gradients of allicin and ajoene running inversely to bioflavonoids. The concentrations used are 50 μg/ml, 25 mg/ml, 12.5 μg/ml, 6.25 μg/ml, 3.125 μg/ml and 1.5625 μg/ml.

130 μl of sterile nutrient broth (Nutrient Broth No. 3 (Sigma 70149)) was added to each well.

50 μl of each microorganism suspended in saline was added to each well (except for the negative control). Each species of microorganism was added to two plates, the first for the individual (control) assays and the second for the matrix study. The plates were incubated at 37.0° C. overnight without agitation.

### Example 5

### Data Collection and Analysis

The plates were scanned individually using a Tecan plate reader at 600 nm and the absorbance recorded in a comma-delimited file for analysis.

The absorbance for each sample was compared to the growth (absorbance) of the control cultures (without treatment) in order to determine the MIC (all values blanked against negative control to negate absorbance of plate and broth). The mean and standard deviation of the triplicate experiments were determined and the means plotted on scatter diagrams for comparison.

The response of the microorganisms to the individual compounds of garlic extracts was dose (concentration) dependent as seen in FIG. 1. The MIC was reached at very low concentrations as reflected in Table 1.

TABLE 1
MICs for AL and AJ as determined
in one example of the studies:

| Organism | Allicin | Ajoene |
|---|---|---|
| Bacillus subtilis | 9.2 μg/ml | 24 μg/ml |
| Staphylococcus aureus | 27.6 μg/ml | 48 μg/ml |
| Escherichia coli | 18.4 μg/ml | 24 μg/ml |
| Pseudomonas aeruginosa | 9.2 μg/ml | 12 μg/ml |
| Klebsiella pneumonia | 46 μg/ml | 12 μg/ml |
| Candida tropicalis | 9.2 μg/ml | 24 μg/ml |

Synergy in antimicrobial activity was determined as any antimicrobial activity that was greater than the additive effect of the individual compounds.

Table 2 shows the presence or absence of synergy in one or more of the concentration combinations between AL, AJ, and the selected bioflavonoids. A full list of concentrations which produced synergy can be found in Table 3 for different combinations between bioflavonoids and garlic extracts. Where synergy was indicated the additive inhibitory effect of the two individual compounds was exceeded by the inhibitory effect expressed by the combination, for each combination this effect is concentration dependent.

TABLE 2

| Garlic Extract | Allicin | | | | Ajoene | | | |
|---|---|---|---|---|---|---|---|---|
| Bioflavonoid | Naringenin | Naringin | Quercetin | Rhoifolin | Naringenin | Naringin | Quercetin | Rhoifolin |
| S. Aureus | Yes | Yes | Yes | Yes | Yes | Yes | Yes | Yes |
| E. coli | Yes | Yes | Yes | Yes | Yes | Yes | Yes | Yes |
| P. aeruginosa | Yes | Yes | Yes | Yes | Yes | Yes | Yes | Yes |
| K. pneumoniae | Yes | Yes | Yes | Yes | Yes | Yes | Yes | Yes |
| C. tropicalis | Yes | Yes | Yes | Yes | Yes | Yes | Yes | Yes |

Table 3 shows the synergy determination for different concentrations of allicin and ajoene, respectively, in combination with four individual bioflavonoids (naringenin, naringin, quercetin and rhoifolin. In Table 3, the results are presented in the sequence allicin or ajoene+Bioflavonoid.

TABLE 3

| | Allicin (μg/ml) | | | | Ajoene (μg/ml) | | | |
|---|---|---|---|---|---|---|---|---|
| Garlic Extract | Naringenin | Naringin | Quercetin | Rhoifolin | Naringenin | Naringin | Quercetin | Rhoifolin |
| Bioflavonoid | (μg/ml) | (μg/ml) | (μg/ml) | (μg/ml) | (μg/ml) | (μg/ml) | (μg/ml) | (μg/ml) |
| S. Aureus | 25+1.5625 | 6.25+6.25, | 3.125+12.5, | 1.5625+25, | 12.5+3.125, | 3.125+12.5, | 3.125+12.5, | 1.5625+25, |
| | | 12.5+3.125, | 6.25+6.25 | 3.125+12.5, | 25+1.5625 | 6.25+6.25, | 6.25+6.25 | 3.125+12.5, |
| | | 25+1.5625 | | 6.25+6.25, | | 12.5+3.125, | | 6.25+6.25, |
| | | | | 12.5+3.125, | | 25+1.5625 | | 12.5+3.125, |
| | | | | 25+1.5625 | | | | 25+1.5625 |
| E. coli | 1.5625+25, | 1.5625+25, | 1.5625+25, | 1.5625+25, | 1.5625+25, | 1.5625+25, | 1.5625+25, | 1.5625+25, |
| | 3.125+12.5, | 3.125+12.5, | 3.125+12.5, | 3.125+12.5, | 3.125+12.5, | 3.125+12.5, | 3.125+12.5, | 3.125+12.5, |
| | 6.25+6.25, | 6.25+6.25, | 6.25+6.25, | 6.25+6.25, | 6.25+6.25, | 6.25+6.25, | 6.25+6.25, | 6.25+6.25, |
| | 12.5+3.125, | 12.5+3.125, | 12.5+3.125, | 12.5+3.125, | 12.5+3.125 | 12.5+3.125, | 12.5+3.125, | 12.5+3125 |
| | 25+1.5625 | 25+1.5625 | 25+1.5625 | 25+1.5625 | | 25+1.5625 | 25+1.5625 | |
| P. aeruginosa | 1.5625+25, | 1.5625+25, | 1.5625+25, | 1.5625+25, | 6.25+6.25 | 6.25+6.25 | 1.5625+25, | 1.5625+25, |
| | 3.125+12.5 | 3125+12.5, | 3.125+12.5, | 3.125+12.5, | | | 3.125+12.5, | 3.125+12.5, |
| | | 12.5+3.125, | 6.25+6.25, | 6.25+6.25, | | | 6.25+6.25, | 6.25+6.25, |
| | | 25+1.5625 | 12.5+3.125, | 12.5+3.125, | | | 12.5+1.5625 | 12.5+3.125, |
| | | | 25+1.5625 | 25+1.5625 | | | | 25+1.5625 |
| K. pneumoniae | 1.5625+25, | 1.5625+25, | 1.5625+25, | 6.25+6.25, | 1.5625+25, | 1.5625+25, | 1.5625+25, | 1.5625+25, |
| | 3.125+12.5, | 3.125+12.5, | 3.125+12.5, | 12.5+3125 | 3.125+12.5, | 3.125+12.5, | 3.125+12.5, | 3.125+12.5, |
| | 6.25+6.25 | 6.25+6.25, | 6.25+6.25, | | 6.25+6.25, | 6.25+6.25, | 6.25+6.25, | 6.25+6.25, |
| | | 12.5+3.125 | 12.5+3.125 | | 12.5+3.125, | 12.5+3.125 | 12.5+3.125, | 12.5+3.125 |
| | | | | | | | 25+1.5625 | |
| C. tropicalis | 25+1.5625 | 25+1.5625 | 3.125+12.5, | 3.125+12.5, | 12.5+3.125, | 1.5625+25, | 1.5625+25, | 12.5+3.125, |
| | | | 6.25+6.25, | 6.25+6.25, | 25+1.5625 | 3.125+12.5, | 3.125+12.5, | 25+1.5625 |
| | | | 12.5+3.125 | 12.5+3.125, | | 6.25+6.25, | 6.25+6.25, | |
| | | | | 25+1.5625 | | 12.5+3.125, | 12.5+3.125, | |
| | | | | | | 25+1.5625 | 25+1.5625 | |

Synergy can be seen in the 20 test parameters with allicin as well as the 20 test parameters with ajoene. This shows a high synergy rate between garlic and bioflavonoids for the organisms tested, in particular with the Gram-negative organisms selected.

Some of the synergistic combinations are more concentration dependant than others, for example as seen in Table 3, C. tropicalis only showed a synergistic effect at one concentration for both allicin+naringin and allicin+naringenin. In contrast, E. coli showed a synergistic effect across all concentration ranges for all bioflavonoids when combined with allicin.

The results of this study suggest that synergy between alternatives may be a good place to look for a solution to the growing problem of antibiotic resistance. The results of this study may provide an answer to this threat as the synergistic effects were particularly high against the Gram-negative organisms tested.

### Example 6

### Synergistic Test among Garlic Extracts, Bioflavonoids and Organic Acids

An experimental matrix was set up to study the synergism among garlic extracts, bioflavonoids and organic acids. The exemplary compounds selected were:
(a) garlic extracts: allicin (Al) and ajoene (Aj);
(b) bioflavonoids: quercetin (Q) and acacetin (Ac); and
(c) organic acids: citric acid (C) and malic acid (M).

The preparation of microbial cultures, compounds, plates were conducted using the procedures as detailed in Examples 2 to 4. Data was collected and analysed as shown in Tables 4 and 5.

Table 4 shows a summary of the absence or presence of synergistic inhibitory effects of different combinations among garlic extracts, bioflavonoids and organic acids against the similar series of microorganisms as stated in Example 2, ie S. aureus, E. coli, P. aeruginosa, K pneumonia, C. tropicalis, with an additional strain of Gram-positive bacteria S. viridans. Table 5 is a full list of concentrations which produced synergy. In Table 5, the results in concentration ratio are presented in the sequence of (allicin or ajoene)+(malic acid or citric acid)+(acacetin or quercetin).

There is a high rate of synergy shown by the combinations of the three active ingredients, ie garlic extract, bioflavonoid and organic acid, with respect to the organisms tested, which include Gram-positive bacteria (S. aureus and S. viridans), Gram-negative bacteria (E. coli, P. aeruginosa and K. pneumonia) and yeast (C. tropicalis).

TABLE 4

| Garlic Extract | Allicin | | | | Ajoene | | | |
|---|---|---|---|---|---|---|---|---|
| Bioflavonoid | Acacetin | | Quercetin | | Acacetin | | Quercetin | |
| Organic Acid | Malic acid | Citric acid | Malic acid | Citric acid | Malic acid | Citric acid | Malic acid | Citric acid |
| S. aureus | Yes | Yes | Yes | Yes | Yes | Yes | Yes | Yes |
| S. viridans | Yes | Yes | Yes | Yes | Yes | Yes | Yes | Yes |
| E. coli | Yes | Yes | Yes | Yes | Yes | Yes | Yes | Yes |
| P. aeruginosa | Yes | Yes | Yes | Yes | Yes | Yes | Yes | Yes |
| K. pneumoniae | Yes | Yes | Yes | Yes | Yes | Yes | Yes | Yes |
| C. tropicalis | Yes | Yes | Yes | Yes | Yes | Yes | Yes | Yes |

TABLE 5

| Garlic | | | | | | | | |
|---|---|---|---|---|---|---|---|---|
| Extract | | | | | | | | |
| Biofla- | Allicin (μg/ml) | | | | Ajoene (μg/ml) | | | |
| vonoid | Acacetin (μg/ml) | | Quercetin (μg/ml) | | Acacetin (μg/ml) | | Quercetin (μg/ml) | |
| Organic | Malic acid | Citric acid | Malic acid | Citric acid | Malic acid | Citric acid | Malic acid | Citric acid |
| Acid | (μg/ml) | (μg/ml) | (μg/ml) | (μg/ml) | (μg/ml) | (μg/ml) | (μg/ml) | (μg/ml) |
| S. | 25+1.5625+ | 25+1.5625+ | 25+1.5625+ | 25+1.5625+ | 25+1.5625+ | 25+1.5625+ | 25+1.5625+ | 25+1.5625+ |
| Aureus | 1.5625, 12.5+ | 1.5625, 12.5+ | 1.5625, 12.5+ | 1.5625, 12.5+ | 1.5625, 12.5+ | 1.5625, 12.5+ | 1.5625, 12.5+ | 1.5625, 12.5+ |
| | 3.125+3.125, | 3.125+3.125, | 3.125+3.125, | 3.125+3.125 | 3.125+3.125, | 3.125+3.125, | 3.125+3.125, | 3.125+3.125, |
| | 6.25+6.25+ | 6.25+6.25+ | 6.25+6.25+ | | 6.25+6.25+ | 6.25+6.25+ | 6.25+6.25+ | 6.25+6.25+ |
| | 6.25, 3.125+ | 6.25, 3.125+ | 6.25 | | 6.25, 3.125+ | 6.25, 3.125+ | 6.25, 3.125+ | 6.25, 3.125+ |
| | 12.5+12.5, | 12.5+12.5, | | | 12.5+12.5, | 12.5+12.5, | 12.5+12.5, | 12.5+12.5 |
| | 1.5625+25+25 | 1.5625+25+25 | | | 1.5625+25+25 | 1.5625+25+25 | 1.5625+25+25 | |
| S. | 25+1.5625+ | 25+1.5625+ | 25+1.5625+ | 25+1.5625+ | 25+1.5625+ | 25+1.5625+ | 25+1.5625+ | 25+1.5625+ |
| viridians | 1.5625, 12.5+ | 1.5625, 12.5+ | 1.5625, 12.5+ | 1.5625, 6.25+ | 1.5625, 12.5+ | 1.5625, 12.5+ | 1.5625, 12.5+ | 1.5625, 12.5+ |
| | 3.125+3.125, | 3.125+3.125 | 3.125+3.125, | 6.25+6.25 | 3.125+3.125, | 3.125+3.125, | 3.125+3.125, | 3.125+3.125, |
| | 6.25+6.25+ | | 6.25+6.25+ | | 6.25+6.25+ | 6.25+6.25+ | 6.25+6.25+ | 6.25+6.25+ |
| | 6.25, 3.125+ | | 6.25 | | 6.25, 3.125+ | 6.25, 3.125+ | 6.25, 3.125+ | 6.25, 3.125+ |
| | 12.5+12.5, | | | | 12.5+12.5, | 12.5+12.5, | 12.5+12.5, | 12.5+12.5, |
| | 1.5625+25+25 | | | | 1.5625+25+25 | 1.5625+25+25 | 1.5625+25+25 | 1.5625+25+25 |
| E. | 25+1.5625+ | 25+1.5625+ | 25+1.5625+ | 25+1.5625+ | 25+1.5625+ | 25+1.5625+ | 25+1.5625+ | 25+1.5625+ |
| coli | 1.5625, 6.25+ | 1.5625, 6.25+ | 1.5625, 12.5+ | 1.5625, 12.5+ | 1.5625, 12.5+ | 1.5625, 12.5+ | 1.5625, 12.5+ | 1.5625, 12.5+ |
| | 6.25+6.25, | 6.25+6.25, | 3.125+3.125, | 3.125+3.125, | 3.125+3.125, | 3.125+3.125, | 3.125+3.125, | 3.125+3.125, |
| | 3.125+12.5+ | 3.125+12.5+ | 6.25+6.25+ | 6.25+6.25+ | 6.25+6.25+ | 6.25+6.25+ | 6.25+6.25+ | 6.25+6.25+ |
| | 12.5, 1.5625+ | 12.5, 1.5625+ | 6.25, 3.125+ | 6.25, 3.125+ | 6.25, 3.125+ | 6.25, 3.125+ | 6.25, 3.125+ | 6.25, 3.125+ |
| | 25+25 | 25+25 | 12.5+12.5, | 12.5+12.5, | 12.5+12.5, | 12.5+12.5, | 12.5+12.5, | 12.5+12.5, |
| | | | 1.5625+25+25 | 1.5625+25+25 | 1.5625+25+25 | 1.5625+25+25 | 1.5625+25+25 | 1.5625+25+25 |
| P. | 25+1.5625+ | 25+1.5625+ | 25+1.5625+ | 25+1.5625+ | 25+1.5625+ | 6.25+6.25+6.25 | 25+1.5625+ | 12.5+3.125+ |
| aeruginosa | 1.5625, 12.5+ | 1.5625 | 1.5625, 12.5+ | 1.5625, 12.5+ | 1.5625, 12.5+ | | 1.5625, 12.5+ | 3.125, 6.25+ |
| | 3.125+3.125, | | 3.125+3.125, | 3.125+3.125 | 3.125+3.125, | | 3.125+3.125, | 6.25+6.25 |
| | 6.25+6.25+ | | 1.5625+25+25 | | 6.25+6.25+6.25, | | 6.25+6.25+ | |
| | 6.25 | | | | 3.125+12.5+ | | 6.25, 3.125+ | |
| | | | | | 12.5 | | 12.5+12.5 | |
| K. | 25+1.5625+ | 25+1.5625+ | 25+1.5625+ | 25+1.5625+ | 25+1.5625+ | 25+1.5625+ | 25+1.5625+ | 25+1.5625+ |
| pneu- | 1.5625, 12.5+ | 1.5625, 12.5+ | 1.5625, 12.5+ | 1.5625, 6.25+ | 1.5625, 12.5+ | 1.5625, 12.5+ | 1.5625, 12.5+ | 1.5625, 12.5+ |
| moniae | 3.125+3.125, | 3.125+3.125, | 3.125+3.125, | 6.25+6.25, | 3.125+3.125, | 3.125+3.125, | 3.125+3.125, | 3.125+3.125, |
| | 6.25+6.25+ | 6.25+6.25+ | 6.25+6.25+ | 3.125+12.5+ | 6.25+6.25+ | 6.25+6.25+ | 6.25+6.25+ | 6.25+6.25+ |
| | 6.25, 3.125+ | 6.25, 3.125+ | 6.25, 3.125+ | 12.5, 1.5625+ | 6.25, 3.125+ | 6.25, 3.125+ | 6.25, 3.125+ | 6.25, 3.125+ |
| | 12.5+12.5, | 12.5+12.5, | 12.5+12.5, | 25+25 | 12.5+12.5, | 12.5+12.5, | 12.5+12.5, | 12.5+12.5, |
| | 1.5625+25+25 | 1.5625+25+25 | 1.5625+25+25 | | 1.5625+25+25 | 1.5625+25+25 | 1.5625+25+25 | 1.5625+25+25 |
| C. | 25+1.5625+ | 25+1.5625+ | 25+1.5625+ | 25+1.5625+ | 25+1.5625+ | 25+1.5625+ | 25+1.5625+ | 25+1.5625+ |
| tropicalis | 1.5625, 12.5+ | 1.5625, 12.5+ | 1.5625, 12.5+ | 1.5625, 12.5+ | 1.5625, 12.5+ | 1.5625, 12.5+ | 1.5625, 12.5+ | 1.5625, 12.5+ |
| | 3.125+3.125, | 3.125+3.125, | 3.125+3.125, | 3.125+3.125, | 3.125+3.125, | 3.125+3.125, | 3.125+3.125, | 3.125+3.125, |
| | 6.25+6.25+ | 6.25+6.25+ | 6.25+6.25+ | 6.25+6.25+ | 6.25+6.25+ | 6.25+6.25+ | 6.25+6.25+ | 6.25+6.25+ |
| | 6.25, 3.125+ | 6.25, 3.125+ | 6.25, 3.125+ | 6.25, 3.125+ | 6.25, 3.125+ | 6.25, 3.125+ | 6.25, 3.125+ | 6.25, 3.125+ |
| | 12.5+12.5, | 12.5+12.5, | 12.5+12.5 | 12.5+12.5, | 12.5+12.5, | 12.5+12.5, | 12.5+12.5, | 12.5+12.5, |
| | 1.5625+25+25 | 1.5625+25+25 | | 1.5625+25+25 | 1.5625+25+25 | 1.5625+25+25 | 1.5625+25+25 | 1.5625+25+25 |

1. An antimicrobial composition comprising:
at least one garlic extract; and
one or more of a bioflavonoid and/or an organic acid.

2. An antimicrobial composition according to claim 1, wherein the composition comprises at least one garlic extract and at least one bioflavonoid.

3. An antimicrobial composition according to claim 2, wherein the concentration ratio of the garlic extract to the bioflavonoid is about 1-16:16-1.

4. An antimicrobial composition according to claim 2, wherein the concentration ratio of the garlic extract to the bioflavonoid is about 1-4:4-1.

5. An antimicrobial composition according to claim 1, wherein the composition comprises at least one garlic extract and at least one organic acid.

6. An antimicrobial composition according to claim 5, wherein the concentration ratio of the garlic extract to the organic acid is about 1-16:16-1.

7. An antimicrobial composition according to claim 5, wherein the concentration ratio of the garlic extract to the organic acid is about 1-4:4-1.

8. An antimicrobial composition according to claim 1, wherein the composition comprises at least one garlic extract, at least one bioflavonoid and at least one organic acid.

9. An antimicrobial composition according to claim 8, wherein the concentration ratio of the garlic extract to the bioflavonoid and to the organic acid is about 1-16:1-16:1-16.

10. An antimicrobial composition according to claim 8, or 9, wherein the concentration ratio of the garlic extract to the bioflavonoid and to the organic acid is about 1-4:1-4:1-4.

11. An antimicrobial composition according to claim 1, wherein the garlic extract is selected from one or more of crude garlic, allicin and ajoene.

12. An antimicrobial composition according to claim 1, wherein the bioflavonoid is selected from one or more of the group consisting of flavones, flavonols, flavanones, flavanone glycosides, flavanonols, flavans and anthocyanidins.

13. An antimicrobial composition according to claim 1, wherein the bioflavonoid is selected from one or more of acacetin, rhoifolin luteolin, apigenin, tangeritin, quercetin, kaempferol, myricetin, fisetin, galangin, isohamnetin, pachypodol, rhamnazin, hesperetin, naringenin, eriodictyol, homoeriodictyol, naringin, hesperidin, neohesperidin, catechin, cardamonin, procyanidin, sillibinin and genistein.

14. An antimicrobial composition according to claim 1, wherein the organic acid is selected from one or more of carboxylic acids and sulfonic acids.

15. An antimicrobial composition according to claim 1, wherein the organic acid is selected from one or more of lactic acid, butyric acid, propionic acid, valeric acid, caproic acid, acetic acid, formic acid, citric acid, oxalic acid, sorbic acid, benzoic acid, caprylic acid and malic acid, or any combination thereof.

16. An antimicrobial composition according to claim 1, further comprising a pharmaceutically acceptable carrier and/or excipient.

17. An antimicrobial composition according to claim 1, wherein the composition is formulated for oral, topical, intravenous, intramuscular, intrarectal (suppository), inhalant, infusion, transdermal, sublingual, subcutaneous or intranasal administration.

18. An antimicrobial composition according to claim 1, wherein the composition is antibacterial.

19. An antimicrobial composition according to claim 1, wherein the composition is antifungal.

20. An antimicrobial composition according to claim 1, wherein the composition is antiparasitic.

21. An antimicrobial composition according to claim 1, wherein the composition is for use in treating or preventing a bacterial infection.

22. An antimicrobial composition for use according to claim 21, wherein the bacterial infection is a Gram-negative bacterial infection.

23. An antimicrobial composition for use according to claim 21, wherein the bacterial infection is a Gram-positive bacterial infection.

24. An antimicrobial composition according to claim 1, wherein the composition is for use in treating or preventing a fungal infection.

25. An antimicrobial composition according to claim 1, wherein the composition is for use in treating or preventing a parasitic infection.

26. A method of treating or preventing a bacterial infection, the method comprising administering an antimicrobial composition according to claim 1 to a subject in need thereof

27. A method of treating or preventing a fungal infection, the method comprising administering an antimicrobial composition according to claim 1 to a subject in need thereof.

28. A method of treating or preventing a parasitic infection, the method comprising administering an antimicrobial composition according to claim 1 to a subject in need thereof.

29. A method of manufacturing a medicament for treating or preventing a bacterial infection in a subject comprising adding the antimicrobial composition of claim 1 to a medicament.

30. A method of manufacturing a medicament for treating or preventing a fungal infection in a subject comprising adding the antimicrobial composition of claim 1 to a medicament.

31. A method of manufacturing a medicament for treating or preventing a fungal infection in a subject comprising adding the antimicrobial composition of claim 1 to a medicament.
